SEQUENCE LISTING (2 sequences)

SEQ ID NO: 1 - DNA, 20 bp, Zea mays
  source 1..20: mol_type = genomic DNA
tggcttttgctgaggggata

SEQ ID NO: 2 - DNA, 36 bp, synthetic construct
  source 1..36: mol_type = other DNA
  modified_base 1^2: mod_base = OTHER; note = phosphorothioate bond
  modified_base 2^3: mod_base = OTHER; note = phosphorothioate bond
  modified_base 34^35: mod_base = OTHER; note = phosphorothioate bond
  modified_base 35^36: mod_base = OTHER; note = phosphorothioate bond
gtaagcgcttacgtaagcgcttacgtaagcgcttac

(19) United States
(12) Patent Application Publication
(10) Pub. No.: US 2025/0230454 A1
(43) Pub. Date: Jul. 17, 2025

(54) PLANT GENOME EDITING METHODS

(71) Applicant: INARI AGRICULTURE TECHNOLOGY, INC., Cambridge, MA (US)

(72) Inventors: Frédéric Van Ex, Kortrijk (BE); Hannes Bart Claeys, Gent (BE); Eveline Neyrinck, De Pinte (BE)

(21) Appl. No.: 18/703,773

(22) PCT Filed: Nov. 8, 2022
(86) PCT No.: PCT/US2022/079441

§ 371 Date: Apr. 23, 2024

Related U.S. Application Data
(60) Provisional application No. 63/263,736, filed on Nov. 8, 2021.

(51) Int. Cl. C12N 15/82; C12N 9/22; C12N 15/11

(52) U.S. Cl. CPC C12N 15/8213; C12N 9/22; C12N 15/11; C12N 15/8205; C12N 15/8207; C12N 2310/20

(57) ABSTRACT

The disclosure relates to methods of producing genome edited plant cells, genome edited plant calli, as well as genome edited plants. The methods comprise first introducing into a plant cell the polypeptide element(s) of a genome-editing system and, subsequently, introducing the polynucleotide element(s) of the genome-editing system into a plant cell that is already expressing the polypeptide element(s).

### CROSS-REFERENCE TO RELATED APPLICATIONS

This International Patent Application claims the benefit of U.S. Provisional Patent Application Nos. 63/263,736, filed Nov. 8, 2021, which is incorporated herein by reference in its entirety.

### SEQUENCE LISTING XML

This application contains a Sequence Listing which has been submitted electronically in XML file format and is hereby incorporated by reference in its entirety. The XML file, created on Nov. 7, 2022, is named P13633WO00.xml and is 4,850 bytes in size.

### BACKGROUND

Methods of using CRISPR, Zinc Finger Nuclease, and Transcription activator like effector Nuclease (TALEN) technology for genome editing in plants are disclosed in US 20150082478, US 2015/0059010A1, and Bortesi et al., 2015, Biotechnology Advances, pp. 41-52, Vol. 33, No. 1. Although genome editing techniques are available for producing targeted insertion of sequences into plants, there remains a need for improved transformation methods that can be used to efficiently edit the genome of a plant.

### SUMMARY

Methods of producing a genome edited plant cell comprising: (a) introducing into a plant cell a polynucleotide encoding one or more polypeptide element(s) of a genome-editing system; (b) selecting for a plant cell that expresses the polypeptide element(s); (c) introducing one or more polynucleotide element(s) of the genome-editing system into the plant cell that expresses the polypeptide element(s); and (d) identifying a genome edited plant cell from the plant cell obtained in step (c) are provided.

Methods of producing a genome edited plant callus comprising: (a) introducing into a plant cell a polynucleotide encoding one or more polypeptide element(s) of a genome-editing system; (b) selecting callus that expresses the polypeptide element(s), wherein the callus is obtained from the plant cell of step (a); (c) introducing into cells of the callus that expresses the polypeptide element(s) one or more polynucleotide elements of the genome-editing system; and (d) identifying a genome edited plant callus comprising a genome edited plant cell from the plant cell obtained in step (c) are provided.

### DETAILED DESCRIPTION

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “expression” refers to the production of a functional end-product (e.g., an mRNA, guide RNA, or a protein) in either precursor or mature form.

As used herein, the terms “include,” “includes,” and “including” are to be construed as at least having the features to which they refer while not excluding any additional unspecified features.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

The term “isolated” as used herein means having been removed from its natural environment.

As used herein, the phrase “operably linked” refers to a juxtaposition wherein the components so described are in a relationship permitting them to function in their intended manner. For instance, a promoter is operably linked to a coding sequence if the promoter affects its transcription or expression.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks. In contrast, some plant cells are not capable of being regenerated to produce plants and are referred to herein as “non-regenerable” plant cells.

As used herein, “selecting” is understood as identifying and isolating or enriching for one or more plant cells having a desired characteristic or property of interest, for example, a selectable marker.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Effective genome editing of a plant cell requires transformation of a progenitor cell with multiple elements that subsequently need to interact. Some elements are expressed proteins, which require time from transfection or transformation to translation. Other elements are nucleic acids like DNA or RNA that have a limited lifetime inside a cell. Provided herein are methods comprising a two-step transformation process for an optimum mixture of all the necessary elements of the genome-editing system at a sufficient concentration. A first transformation inserts the polypeptide elements, such as nucleases. Later, nucleic acids are introduced into the same cells. The overall efficiency of genome editing is improved over a one-step transformation method that introduces all the elements of the genome-editing system at one time.

Provided herein are methods of producing a genome edited plant cell comprising the steps of: (a) introducing into a plant cell a polynucleotide encoding one or more polypeptide element(s) of a genome-editing system; (b) selecting for a plant cell that expresses the polypeptide element(s); (c) introducing one or more polynucleotide element(s) of the genome-editing system into the plant cell that expresses the polypeptide element(s); and (d) identifying a genome edited plant cell from the plant cell obtained in step (c).

Also provided are methods of producing a genome edited plant callus comprising the steps of: (a) introducing into a plant cell a polynucleotide encoding one or more polypeptide element(s) of a genome-editing system; (b) selecting callus that expresses the polypeptide element(s), wherein the callus is obtained from the plant cell of step (a); (c) introducing into cells of the callus that expresses the polypeptide element(s) one or more polynucleotide elements of the genome-editing system; and (d) identifying a genome edited plant callus comprising a genome edited plant cell from the plant cell obtained in step (c).

In certain embodiments, methods provided herein can include the additional step of growing or regenerating a genome edited plant from a genome edited plant cell or a genome edited plant callus. In certain embodiments, callus is produced from the plant cell, and plantlets and plants produced from such callus. In other embodiments, whole seedlings or plants are grown directly from the plant cell without a callus stage. Methods for obtaining regenerable plant structures and regenerating plants from plant cells or regenerable plant structures can be adapted from published procedures (Roest and Gilissen, Acta Bot. Neerl., 1989, 38(1), 1-23; Bhaskaran and Smith, Crop Sci. 30(6):1328-1337; Ikeuchi et al., Development, 2016, 143: 1442-1451). Methods for obtaining regenerable plant structures and regenerating plants from plant cells or regenerable plant structures can also be adapted from US Patent Application Publication No. 20170121722, which is incorporated herein by reference in its entirety and specifically with respect to such disclosure.

The polynucleotide encoding the polypeptide element(s) of a genome-editing system and the polynucleotide element(s) of the genome-editing system are not introduced simultaneously, but rather a plant cell is first established which expresses the polypeptide element(s). In certain embodiments, the plant cell that expresses the polypeptide element(s) has not been obtained from a regenerated plant that expresses the polypeptide element(s). In certain embodiments, the time between the introducing of the polynucleotide encoding the polypeptide element(s) of the genome-editing system and the introducing of the polynucleotide element(s) of the genome-editing system is at least about 12 hours, about 24 hours, about 36 hours, about 48 hours, about 60 hours, about 72 hours, about 84 hours, about 96 hours, about 108 hours, about 120 hours, about 132 hours, about 144 hours, about 1 week, about 2 weeks, about 3 weeks, about 4 weeks, about 5 weeks, or about 6 weeks. In certain embodiments, the time between the introducing of the polynucleotide encoding the polypeptide element(s) of the genome-editing system and the introducing of the polynucleotide element(s) of the genome-editing system is from about from about 72 hours to about 108 hours, from about 72 hours to about 120 hours, from about 72 hours to about 132 hours, from about 72 hours to about 144 hours, from about 84 hours to about 108 hours, from about 84 hours to about 120 hours, from about 84 hours to about 132 hours, from about 84 hours to about 144 hours, from about 96 hours to about 108 hours, from about 96 hours to about 120 hours, from about 96 hours to about 132 hours, or from about 96 hours to about 144 hours. In certain embodiments, the time between the introducing of the polynucleotide encoding the polypeptide element(s) of the genome-editing system and the introducing of the polynucleotide element(s) of the genome-editing system is from about 1 week to about 6 weeks, from about 2 weeks to about 6 weeks, from about 3 weeks to about 6 weeks, from about 4 weeks to about 6 weeks, from about 1 week to about 5 weeks, from about 2 weeks to about 5 weeks, from about 3 weeks to about 5 weeks, from about 4 weeks to about 5 weeks, from about 1 week to about 4 weeks, from about 2 weeks to about 4 weeks, or from about 3 weeks to about 4 weeks.

Gene editing molecules of use in methods provided herein include molecules capable of introducing a double-strand break (“DSB”) or single-strand break (“SSB”) in double-stranded DNA, such as in genomic DNA or in a target gene located within the genomic DNA as well as accompanying guide RNA or donor DNA template polynucleotides. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a type II Cas nuclease, a Cas9, a nCas9 nickase, a type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for an RNA-guided nuclease, or a DNA encoding a gRNA for an RNA-guided nuclease; (d) donor DNA template polynucleotides; and (e) other DNA templates (dsDNA, ssDNA, or combinations thereof) suitable for insertion at a break in genomic DNA (e.g., by non-homologous end joining (NHEJ) or microhomology-mediated end joining (MMEJ).

CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, one or more CRISPR endonucleases with unique PAM recognition sites can be used. Guide RNAs (sgRNAs or crRNAs and a tracrRNA) to form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12a proteins. In some instances, Cas12a can also recognize a 5′-CTA PAM motif Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites. Introduction of one or more of a wide variety of CRISPR guide RNAs that interact with CRISPR endonucleases integrated into a plant genome or otherwise provided to a plant is useful for genetic editing for providing desired phenotypes or traits, for trait screening, or for gene editing mediated trait introgression (e.g., for introducing a trait into a new genotype without backcrossing to a recurrent parent or with limited backcrossing to a recurrent parent). Multiple endonucleases can be provided in expression cassettes with the appropriate promoters to allow multiple genome site editing.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Other CRISPR nucleases useful for editing genomes include Cas12b and Cas12c (see Shmakov et al. (2015) Mol. Cell, 60:385-397; Harrington et al. (2020) Molecular Cell doi:10.1016/j.molcel.2020.06.022) and CasX and CasY (see Burstein et al. (2016) Nature, doi:10.1038/nature21059; Harrington et al. (2020) Molecular Cell doi:10.1016/j.molcel.2020.06.022), or Cas12j (Pausch et al, (2020) Science 10.1126/science.abb1400). Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). All of the patent publications referenced in this paragraph are incorporated herein by reference in their entirety.

In certain embodiments, an RNA-guided endonuclease that leaves a blunt end following cleavage of the target site is used. Blunt-end cutting RNA-guided endonucleases include Cas9, Cas12c, and Cas 12h (Yan et al., 2019). In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e.

The methods can also use sequence-specific endonucleases or sequence-specific endonucleases and guide RNAs that cleave a single DNA strand in a dsDNA target site. Such cleavage of a single DNA strand in a dsDNA target site is also referred to herein and elsewhere as “nicking” and can be affected by various “nickases” or systems that provide for nicking. Nickases that can be used include nCas9 (Cas9 comprising a D10A amino acid substitution), nCas12a (e.g., Cas12a comprising an R1226A amino acid substitution; Yamano et al., 2016), Cas12i (Yan et al. 2019), a zinc finger nickase e.g., as disclosed in Kim et al., 2012), a TALE nickase (e.g., as disclosed in Wu et al., 2014), or a combination thereof. In certain embodiments, systems that provide for nicking can comprise a Cas nuclease (e.g., Cas9 and/or Cas12a) and guide RNA molecules that have at least one base mismatch to DNA sequences in the target editing site (Fu et al., 2019). In certain embodiments, genome modifications can be introduced into the target editing site by creating single stranded breaks (i.e., “nicks”) in genomic locations separated by no more than about 10, 20, 30, 40, 50, 60, 80, 100, 150, or 200 base pairs of DNA. In certain illustrative and non-limiting embodiments, two nickases (i.e., a CAS nuclease which introduces a single stranded DNA break including nCas9, nCas12a, Cas12i, Cas 12j, zinc finger nickases, TALE nickases, combinations thereof, and the like) or nickase systems can directed to make cuts to nearby sites separated by no more than about 10, 20, 30, 40, 50, 60, 80 or 100 base pairs of DNA. In instances where an RNA guided nickase and an RNA guide are used, the RNA guides are adjacent to PAM sequences that are sufficiently close (i.e., separated by no more than about 10, 20, 30, 40, 50, 60, 80, 100, 150, or 200 base pairs of DNA). For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least 16 or 17 nucleotides of gRNA sequence are required by Cas9 for DNA cleavage to occur; for Cpf1 at least 16 nucleotides of gRNA sequence are needed to achieve detectable DNA cleavage and at least 18 nucleotides of gRNA sequence were reported necessary for efficient DNA cleavage in vitro; see Zetsche et al. (2015) Cell, 163:759-771. In practice, guide RNA sequences are generally designed to have a length of 17-24 nucleotides (frequently 19, 20, or 21 nucleotides) and exact complementarity (i.e., perfect base-pairing) to the targeted gene or nucleic acid sequence; guide RNAs having less than 100% complementarity to the target sequence can be used (e.g., a gRNA with a length of 20 nucleotides and 1-4 mismatches to the target sequence) but can increase the potential for off-target effects. The design of effective guide RNAs for use in plant genome editing is disclosed in US Patent Application Publication 2015/0082478 A1, the entire specification of which is incorporated herein by reference. More recently, efficient gene editing has been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing); see, for example, Cong et al. (2013) Science, 339:819-823; Xing et al. (2014) BMC Plant Biol., 14:327-340. Chemically modified sgRNAs have been demonstrated to be effective in genome editing; see, for example, Hendel et al. (2015) Nature Biotechnol., 985-991. The design of effective gRNAs for use in plant genome editing is disclosed in US Patent Application Publication 2015/0082478 A1, the entire specification of which is incorporated herein by reference.

Genomic DNA may also be modified via base editing. Both adenine base editors (ABE) which convert A/T base pairs to G/C base pairs in genomic DNA as well as cytosine base pair editors (CBE) which effect C to T substitutions can be used in certain embodiments of the methods provided herein. In certain embodiments, useful ABE and CBE can comprise genome site specific DNA binding elements (e.g., RNA-dependent DNA binding proteins including catalytically inactive Cas9 and Cas12 proteins or Cas9 and Cas12 nickases) operably linked to adenine or cytidine deaminases and used with guide RNAs which position the protein near the nucleotide targeted for substitution. Suitable ABE and CBE disclosed in the literature (Kim, Nat Plants, 2018 March; 4(3):148-151) can be adapted for use in the methods set forth herein. In certain embodiments, a CBE can comprise a fusion between a catalytically inactive Cas9 (dCas9) RNA dependent DNA binding protein fused to a cytidine deaminase which converts cytosine (C) to uridine (U) and selected guide RNAs, thereby effecting a C to T substitution; see Komor et al. (2016) Nature, 533:420-424. In other embodiments, C to T substitutions are effected with Cas9 nickase [Cas9n(D10A)] fused to an improved cytidine deaminase and optionally a bacteriophage Mu dsDNA (double-stranded DNA) end-binding protein Gam; see Komor et al., Sci Adv. 2017 August; 3(8):eaao4774. In other embodiments, adenine base editors (ABEs) comprising an adenine deaminase fused to catalytically inactive Cas9 (dCas9) or a Cas9 D10A nickase can be used to convert A/T base pairs to G/C base pairs in genomic DNA (Gaudelli et al., (2017) Nature 551(7681):464-471.

The polynucleotide element(s) of the genome-editing system can include a donor template polynucleotide. In certain embodiments, at least one double-stranded break (DSB) is effected at a precisely determined site in the plant genome, for example by means of an RNA-guided nuclease and guide RNAs, and a nucleotide sequence encoded by a donor polynucleotide is heterologously integrated at the site of the DSB (or between two DSBs). In embodiments, the donor polynucleotide includes single-stranded DNA, optionally including chemical modifications. In other embodiments, the donor polynucleotide includes double-stranded DNA, optionally including chemical modifications. In some embodiment the donor polynucleotide includes both DNA and RNA, for example as a duplex formed by a DNA strand and an RNA strand.

In embodiments, the donor DNA template molecule includes chemically modified nucleotides (see, e.g., the various modifications of internucleotide linkages, bases, and sugars described in Verma and Eckstein (1998) Annu. Rev. Biochem., 67:99-134); in embodiments, the naturally occurring phosphodiester backbone of the donor DNA template molecule is partially or completely modified with phosphorothioate, phosphorodithioate, or methylphosphonate internucleotide linkage modifications, or the donor DNA template molecule includes modified nucleoside bases or modified sugars, or the donor DNA template molecule is labelled with a fluorescent moiety (e.g., fluorescein or rhodamine or a fluorescent nucleoside analogue) or other detectable label (e.g., biotin or an isotope). In another embodiment, the donor DNA template molecule contains secondary structure that provides stability or acts as an aptamer.

Donor template molecules used in the methods provided herein include DNA molecules comprising, from 5′ to 3′, a first homology arm, a replacement DNA, and a second homology arm, wherein the homology arms containing sequences that are partially or completely homologous to genomic DNA (gDNA) sequences flanking a target site-specific endonuclease cleavage site in the gDNA. In certain embodiments, the replacement DNA can comprise an insertion, deletion, or substitution of 1 or more DNA base pairs relative to the target gDNA. In one embodiment, the donor DNA template molecule is double-stranded and perfectly base-paired through all or most of its length, with the possible exception of any unpaired nucleotides at either terminus or both termini. In another embodiment, the donor DNA template molecule is double-stranded and includes one or more non-terminal mismatches or non-terminal unpaired nucleotides within the otherwise double-stranded duplex. In an embodiment, the donor DNA template molecule that is integrated at the site of at least one double-strand break (DSB) includes between 2-20 nucleotides in one (if single-stranded) or in both strands (if double-stranded), e. g., 1, 2, 3, 4, 5, 6, 7, 8, 9, 10, 11, 12, 13, 14, 15, 16, 17, 18, 19, or 20 nucleotides on one or on both strands, each of which can be base-paired to a nucleotide on the opposite strand of the targeted integration site (in the case of a perfectly base-paired double-stranded polynucleotide molecule). Such donor DNA templates can be integrated in genomic DNA containing blunt and/or staggered double stranded DNA breaks by homology-directed repair (HDR) or microhomology-mediated end joining (MMEJ). In certain embodiments, a donor DNA template homology arm can be about 20, 50, 100, 200, 400, or 600 to about 800, or 1000 base pairs in length. In certain embodiments, a donor DNA template molecule can be delivered to a plant cell in a circular (e.g., a plasmid or a viral vector including a geminivirus vector) or a linear DNA molecule. In certain embodiments, a circular or linear DNA molecule that is used can comprise a modified donor DNA template molecule comprising, from 5′ to 3′, a first copy of the target sequence-specific endonuclease cleavage site sequence, the first homology arm, the replacement DNA, the second homology arm, and a second copy of the target sequence-specific endonuclease cleavage site sequence. In other embodiments, DNA templates suitable for NHEJ insertion will lack homology arms that are partially or completely homologous to genomic DNA (gDNA) sequences flanking a target site-specific endonuclease cleavage site in the gDNA. Donor DNA templates can be synthesized either chemically or enzymatically (e.g., in a polymerase chain reaction (PCR)).

Substitution is optionally by way of homology directed repair (HDR) wherein a donor template polynucleotide comprising replacement DNA used to substitute genomic DNA targeted by a double-stranded break inducing agent. In certain embodiments, HDR-mediated substitution is facilitated by expressing an exonuclease (e.g., a bacteriophage lambda exonuclease), a single-stranded DNA annealing protein (SSAP; e.g., bacteriophage lambda beta SSAP protein), and a single stranded DNA binding protein (SSB; e.g., E. coli SSB) essentially as set forth in US Patent Application Publication 20200407754, which is incorporated herein by reference in its entirety. A DNA sequence encoding a localization signal (NLS; e.g., tobacco c2 NLS) is fused in-frame to the DNA sequences encoding the exonuclease, the SSAP protein, and the SSB. In certain embodiments, a DNA sequence encoding the c2 NLS-Exo, c2 NLS lambda beta SSAP, and c2 NLS-SSB fusion proteins that are set forth in SEQ ID NO: 135, SEQ ID NO: 134, and SEQ ID NO: 133 of US Patent Application Publication 20200407754, respectively, and incorporated herein by reference in its entirety is used. In certain embodiments, DNA sequences encoding the NLS-Exo, NLS-SSAP, and NLS-SSB fusion proteins are operably linked to a promoter (e.g., OsUBI1, ZmUBI1, OsACT promoter) and a polyadenylation site (e.g., OsUbi1, ZmUBI1, OsACT polyadenylation site), to provide the exonuclease, SSAP, and SSB plant expression cassettes. The donor DNA template polynucleotide comprising the replacement DNA will comprise homology arms to the target DNA adjacent to the insertion site in the target genomic DNA.

In general, a donor polynucleotide including a template encoding a nucleotide change over a region of less than about 50 nucleotides is conveniently provided in the form of single-stranded DNA; larger donor templates (e.g., more than 100 nucleotides) are often conveniently provided as double-stranded DNAs. Thus in some embodiments, the donor polynucleotide is about 25 nucleotides, 50 nucleotides, 60 nucleotides, 70 nucleotides 80 nucleotides, 90 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 1200 nucleotides, 1500 nucleotides, 1800 nucleotides, 2000 nucleotides, 2500 nucleotides, 3000 nucleotides, 5000 nucleotides, 10,000 nucleotides, or more (such as about 25-200 nucleotides, 50-300 nucleotides, 100-500 nucleotides, 200-800 nucleotides, 700-2000 nucleotides, 1000-2500 nucleotides, 2000-5000 nucleotides, 4000-8000 nucleotides, or 6000-10,000 nucleotides).

Various treatments can be used for delivery of the polynucleotide encoding the polypeptide element(s) of the genome-editing system and polynucleotide element(s) of the genome-editing system to a plant cell.

In certain embodiments, one or more treatments is employed to deliver the gene editing or other molecules (e.g., comprising a polynucleotide, polypeptide or combination thereof) into a eukaryotic or plant cell, e.g., through barriers such as a cell wall, a plasma membrane, a nuclear envelope, and/or other lipid bilayer. In certain embodiments, a polynucleotide-, polypeptide-, or RNP (ribonucleoprotein)-containing composition comprising the molecules are delivered directly, for example by direct contact of the composition with a plant cell. Aforementioned compositions can be provided in the form of a liquid, a solution, a suspension, an emulsion, a reverse emulsion, a colloid, a dispersion, a gel, liposomes, micelles, an injectable material, an aerosol, a solid, a powder, a particulate, a nanoparticle, or a combination thereof can be applied directly to a plant, plant part, plant cell, or plant explant (e.g., through abrasion or puncture or otherwise disruption of the cell wall or cell membrane, by spraying or dipping or soaking or otherwise directly contacting, by microinjection). For example, a plant cell or plant protoplast is soaked in a liquid genome editing molecule-containing composition. In certain embodiments, the composition is delivered using negative or positive pressure, for example, using vacuum infiltration or application of hydrodynamic or fluid pressure. In certain embodiments, the composition is introduced into a plant cell or plant protoplast, e.g., by microinjection or by disruption or deformation of the cell wall or cell membrane, for example by physical treatments such as by application of negative or positive pressure, shear forces, or treatment with a chemical or physical delivery agent such as surfactants, liposomes, or nanoparticles; see, e.g., delivery of materials to cells employing microfluidic flow through a cell-deforming constriction as described in US Published Patent Application 2014/0287509, incorporated by reference in its entirety herein. Other techniques useful for delivering the composition to a eukaryotic cell, plant cell or plant protoplast include: ultrasound or sonication; vibration, friction, shear stress, vortexing, cavitation; centrifugation or application of mechanical force; mechanical cell wall or cell membrane deformation or breakage; enzymatic cell wall or cell membrane breakage or permeabilization; abrasion or mechanical scarification (e.g., abrasion with carborundum or other particulate abrasive or scarification with a file or sandpaper) or chemical scarification (e.g., treatment with an acid or caustic agent); and electroporation. In certain embodiments, the composition is provided by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of the plant cell or plant protoplast with a polynucleotide encoding the genome editing molecules (e.g., RNA dependent DNA endonuclease, RNA dependent DNA binding protein, RNA dependent nickase, ABE, or CBE, and/or guide RNA); see, e.g., Broothaerts et al. (2005) Nature, 433:629-633). Any of these techniques or a combination thereof are alternatively employed on the plant explant, plant part or tissue or intact plant (or seed) from which a plant cell is optionally subsequently obtained or isolated; in certain embodiments, the composition is delivered in a separate step after the plant cell has been isolated.

In certain embodiments, the polynucleotide encoding one or more polypeptide element(s) of the genome-editing system is introduced by Agrobacterium-mediated transformation and the polynucleotide element(s) of the genome-editing system are introduced by biolistics. In certain embodiments, the polynucleotide encoding one or more polypeptide element(s) of the genome-editing system is introduced by biolistic and the polynucleotide element(s) of the genome-editing system are introduced by biolistics.

The polynucleotide encoding one or more polypeptide element(s) of the genome-editing system can include a nucleotide sequence for a selectable marker, which can be used to select a plant cell expressing the polypeptide(s). The polynucleotide element(s) of the genome-editing system also can include a nucleotide sequence for a selectable marker.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), yellow-green (mNeonGreen), red (RFP; mScarlet), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as glufosinate ammonium, bromoxynil, imidazolinones, and 2,4-dichlorophenoxyacetate (2,4-D). See for example, Yarranton, Curr Opin Biotech (1992) 3:506-1 1; Christopherson et al, Proc. Natl. Acad. Sci. USA (1992) 89:6314-8; Yao et al, Cell (1992) 71:63-72; Reznikoff, Mol Microbiol (1992) 6:2419-22; Hu et al, Cell (1987) 48:555-66; Brown et al, Cell (1987) 49:603-12; Figge et al, Cell (1988) 52:713-22; Deuschle et al, Proc. Natl. Acad. Sci. USA (1989) 86:5400-4; Fuerst et al, Proc. Natl. Acad. Sci. USA (1989) 86:2549-53; Deuschle et al, Science (1990) 248:480-3; Gossen, (1993) Ph.D. Thesis, University of Heidelberg; Reines et al, Proc. Natl. Acad. Sci. USA (1993) 90: 1917-21; Labow et al, Mol Cell Biol (1990) 10:3343-56; Zambretti et al, Proc. Natl. Acad. Sci. USA (1992) 89:3952-6; Bairn et al, Proc. Natl. Acad. Sci. USA (1991) 88:5072-6; Wyborski et al, Nucleic Acids Res (1991) 19:4647-53; Hillen and Wissman, Topics Mol Struc Biol (1989) 10: 143-62; Degenkolb et al, Antimicrob Agents Chemother (1991) 35: 1591-5; Kleinschnidt et al, Biochemistry (1988) 27: 1094-104; Bonin, (1993) Ph.D. Thesis, University of Heidelberg; Gossen et al, Proc. Natl. Acad. Sci. USA (1992) 89:5547-51; Oliva et al, Antimicrob Agents Chemother (1992) 36:913-9; Hlavka et al, Handbook of Experimental Pharmacology, (1985) Vol. 78 (Springer-Verlag, Berlin); Gill et al, Nature (1988) 334:721-4.

Various selection procedures for the cells based on the selectable marker can be used, depending on the nature of the marker gene. In certain embodiments, use is made of a selectable marker, i.e., a marker which allows a direct selection of the cells based on the expression of the marker. A selectable marker can confer positive or negative selection and is conditional or non-conditional on the presence of external substrates (Miki et al. 2004, 107(3): 193-232). Most commonly, antibiotic or herbicide resistance genes are used as a marker, whereby selection is be performed by growing the engineered plant material on media containing an inhibitory amount of the antibiotic or herbicide to which the marker gene confers resistance. Examples of such genes are genes that confer resistance to antibiotics, such as hygromycin (hpt) and kanamycin (nptII), and genes that confer resistance to herbicides, such as phosphinothricin (bar), chlorsulfuron (als), aroA, glyphosate acetyl transferase (GAT) genes, phosphinothricin acetyl transferase (PAT) genes from Streptomyces species, and ACCase inhibitor-encoding genes. Detoxifying genes can also be used as a marker, with examples including an enzyme encoding a phosphinothricin acetyltransferase and hydroxyphenylpyruyate dioxygenase (HPPD) inhibitors.

Transformed plants and plant cells may also be identified by screening for the activities of a visible marker, typically an enzyme capable of processing a colored substrate (e.g., the 0-glucuronidase, luciferase, B or CI genes). Such selection and screening methodologies are well known to those skilled in the art.

In certain embodiment, a heterogeneous population of plant cells having a target gene edit or genome edit (or seedlings or plants grown or regenerated therefrom) is exposed to conditions permitting expression of the phenotype of interest; e.g., selection for herbicide resistance can include exposing the population of plant cells having a target gene edit or genome edit (or seedlings or plants grown or regenerated therefrom) to an amount of herbicide or other substance that inhibits growth or is toxic, allowing identification and selection of those resistant plant cells (or seedlings or plants) that survive treatment.

In some embodiments, one or more polynucleotides or vectors driving expression of one or more genome editing molecules are introduced into a plant cell. In certain embodiments, a polynucleotide vector comprises a regulatory element such as a promoter operably linked to one or more polynucleotides encoding genome editing molecules. In such embodiments, expression of these polynucleotides can be controlled by selection of the appropriate promoter, particularly promoters functional in a eukaryotic cell (e.g., plant cell); useful promoters include constitutive, conditional, inducible, and temporally or spatially specific promoters (e.g., a tissue specific promoter, a developmentally regulated promoter, or a cell cycle regulated promoter). Developmentally regulated promoters that can be used in plant cells include Phospholipid Transfer Protein (PLTP), fructose-1,6-bisphosphatase protein, NAD(P)-binding Rossmann-Fold protein, adipocyte plasma membrane-associated protein-like protein, Rieske [2Fe-2S] iron-sulfur domain protein, chlororespiratory reduction 6 protein, D-glycerate 3-kinase, chloroplastic-like protein, chlorophyll a-b binding protein 7, chloroplastic-like protein, ultraviolet-B-repressible protein, Soul heme-binding family protein, Photosystem I reaction center subunit psi-N protein, and short-chain dehydrogenase/reductase protein that are disclosed in US Patent Application Publication No. 20170121722, which is incorporated herein by reference in its entirety and specifically with respect to such disclosure. In certain embodiments, the promoter is operably linked to nucleotide sequences encoding multiple guide RNAs, wherein the sequences encoding guide RNAs are separated by a cleavage site such as a nucleotide sequence encoding a microRNA recognition/cleavage site or a self-cleaving ribozyme (see, e.g., Ferré-D'Amaré and Scott (2014) Cold Spring Harbor Perspectives Biol., 2:a003574). In certain embodiments, the promoter is an RNA polymerase III promoter operably linked to a nucleotide sequence encoding one or more guide RNAs. In certain embodiments, the RNA polymerase III promoter is a plant U6 spliceosomal RNA promoter, which can be native to the genome of the plant cell or from a different species, e.g., a U6 promoter from maize, tomato, or soybean such as those disclosed U.S. Patent Application Publication 2017/0166912, or a homologue thereof, in an example, such a promoter is operably linked to DNA sequence encoding a first RNA molecule including a Cas12a gRNA followed by an operably linked and suitable 3′ element such as a U6 poly-T terminator. In another embodiment, the RNA polymerase III promoter is a plant U3, 7SL (signal recognition particle RNA), U2, or U5 promoter, or chimerics thereof, e.g., as described in U.S. Patent Application Publication 20170166912. In certain embodiments, the promoter operably linked to one or more polynucleotides is a constitutive promoter that drives gene expression in eukaryotic cells (e.g., plant cells). In certain embodiments, the promoter drives gene expression in the nucleus or in an organelle such as a chloroplast or mitochondrion. Examples of constitutive promoters for use in plants include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and the nopaline synthase (NOS) and octopine synthase (OCS) promoters from Agrobacterium tumefaciens. In certain embodiments, the promoter operably linked to one or more polynucleotides encoding elements of a genome-editing system is a promoter from figwort mosaic virus (FMV), a RUBISCO promoter, or a pyruvate phosphate dikinase (PPDK) promoter, which is active in photosynthetic tissues. Other contemplated promoters include cell-specific or tissue-specific or developmentally regulated promoters, for example, a promoter that limits the expression of the nucleic acid targeting system to germline or reproductive cells (e.g., promoters of genes encoding DNA ligases, recombinases, replicases, or other genes specifically expressed in germline or reproductive cells). In certain embodiments, the genome alteration is limited only to those cells from which DNA is inherited in subsequent generations, which is advantageous where it is desirable that expression of the genome-editing system be limited in order to avoid genotoxicity or other unwanted effects. All of the patent publications referenced in this paragraph are incorporated herein by reference in their entirety.

Expression vectors or polynucleotides provided herein may contain a DNA segment near the 3′ end of an expression cassette that acts as a signal to terminate transcription and directs polyadenylation of the resultant mRNA and may also support promoter activity. Such a 3′ element is commonly referred to as a “3′-untranslated region” or “3′-UTR” or a “polyadenylation signal.” In some cases, plant gene-based 3′ elements (or terminators) consist of both the 3′-UTR and downstream non-transcribed sequence (Nuccio et al., 2015). Useful 3′ elements include: Agrobacterium tumefaciens nos 3′, tml 3′, tmr 3′, tms 3′, ocs 3′, and tr7 3′ elements disclosed in U.S. Pat. No. 6,090,627, incorporated herein by reference, and 3′ elements from plant genes such as the heat shock protein 17, ubiquitin, and fructose-1,6-biphosphatase genes from wheat (Triticum aestivum), and the glutelin, lactate dehydrogenase, and beta-tubulin genes from rice (Oryza sativa), disclosed in US Patent Application Publication 2002/0192813 A1. All of the patent publications referenced in this paragraph are incorporated herein by reference in their entireties.

Target plants suitable for use in the methods provided herein include plants and plant cells of any species of interest, including dicots and monocots. Plants of interest include row crop plants, fruit-producing plants and trees, vegetables, trees, and ornamental plants including ornamental flowers, shrubs, trees, groundcovers, and turf grasses. Examples of commercially important cultivated crops, trees, and plants include: alfalfa (Medicago sativa), almonds (Prunus dulcis), apples (Malus x domestica), apricots (Prunus armeniaca, P. brigantine, P. mandshurica, P. mume, P. sibirica), Asparagus (Asparagus officinalis), bananas (Musa spp.), barley (Hordeum vulgare), beans (Phaseolus spp.), blueberries and cranberries (Vaccinium spp.), cacao (Theobroma cacao), canola and rapeseed or oilseed rape, (Brassica napus), carnation (Dianthus caryophyllus), carrots (Daucus carota sativus), cassava (Manihot esculentum), cherry (Prunus avium), chickpea (Cider arietinum), chicory (Cichorium intybus), chili peppers and other Capsicum peppers (Capsicum annuum, C. frutescens, C. chinense, C. pubescens, C. baccatum), chrysanthemums (Chrysanthemum spp.), coconut (Cocos nucifera), coffee (Coffea spp. including Coffea arabica and Coffea canephora), cotton (Gossypium hirsutum L.), cowpea (Vigna unguiculata), cucumber (Cucumis sativus), currants and gooseberries (Ribes spp.), eggplant or aubergine (Solanum melongena), Eucalyptus (Eucalyptus spp.), flax (Linum usitatissumum L.), geraniums (Pelargonium spp.), grapefruit (Citrus x paradisi), grapes (Vitus spp.) including wine grapes (Vitus vinfera), guava (Psidium guajava), hops (Humulus lupulus), hemp and Cannabis (Cannabis sativa and Cannabis spp.), irises (Iris spp.), lemon (Citrus limon), lettuce (Lactuca sativa), limes (Citrus spp.), maize (Zea mays L.), mango (Mangifera indica), mangosteen (Garcinia mangostana), melon (Cucumis melo), millets (Setaria spp, Echinochloa spp, Eleusine spp, Panicum spp., Pennisetum spp.), oats (Avena sativa), oil palm (Ellis quineensis), olive (Olea europaea), onion (Allium cepa), orange (Citrus sinensis), Papaya (Carica papaya), peaches and nectarines (Prunus persica), pear (Pyrus spp.), pea (Pisa sativum), peanut (Arachis hypogaea), peonies (Paeonia spp.), petunias (Petunia spp.), pineapple (Ananas comosus), plantains (Musa spp.), plum (Prunus domestica), poinsettia (Euphorbia pulcherrima), Polish canola (Brassica rapa), poplar (Populus spp.), potato (Solanum tuberosum), pumpkin (Cucurbita pepo), rice (Oryza sativa L.), roses (Rosa spp.), rubber (Hevea brasiliensis), rye (Secale cereale), safflower (Carthamus tinctorius L), Sesame seed (Sesame indium), Sorghum (Sorghum bicolor), soybean (Glycine max L.), squash (Cucurbita pepo), strawberries (Fragaria spp., Fragaria x ananassa), sugar beet (Beta vulgaris), sugarcanes (Saccharum spp.), sunflower (Helianthus annus), sweet potato (Ipomoea batatas), tangerine (Citrus tangerina), tea (Camellia sinensis), tobacco (Nicotiana tabacum L.), tomato (Lycopersicon esculentum), tulips (Tulipa spp.), turnip (Brassica rapa rapa), walnuts (Juglans spp. L.), watermelon (Citrulus lanatus), wheat (Tritium aestivum), and yams (Discorea spp.). In certain embodiments, the plant cell is a maize plant cell or a soybean plant cell.

### EMBODIMENTS

The following numbered embodiments also form part of the present disclosure:
1. A method of producing a genome edited plant cell comprising: (a) introducing into a plant cell a polynucleotide encoding one or more polypeptide element(s) of a genome-editing system; (b) selecting for a plant cell that expresses the polypeptide element(s); (c) introducing one or more polynucleotide element(s) of the genome-editing system into the plant cell that expresses the polypeptide element(s); and (d) identifying a genome edited plant cell from the plant cell obtained in step (c).
2. The method of embodiment 1, further comprising the step of regenerating a genome edited plant from the genome edited plant cell of step (d).
3. The method of embodiment 1 or embodiment 2, wherein the plant cell that expresses the polypeptide element(s) in step (c) has not been obtained from a regenerated plant that expresses the polypeptide element(s).
4. The method of any one of embodiments 1 to 3, wherein the polypeptide element of the genome-editing system comprises an RNA-guided nuclease.
5. The method of any one of embodiments 1 to 4, wherein the polynucleotide element of the genome-editing system comprises a donor template polynucleotide.
6. The method of any one of embodiments 1 to 5, wherein the polynucleotide element of the genome-editing system comprises a guide RNA (gRNA) for use with the RNA-guided nuclease, or a DNA encoding a gRNA for use with the RNA-guided nuclease.
7. The method of any one of embodiments 1 to 6, wherein the introducing of step (a) is by Agrobacterium-mediated transformation and optionally wherein the introducing of step (c) is by biolistics.
8. The method of any one of embodiments 1 to 7, wherein the introducing of step (c) is by biolistics.
9. The method of any one of embodiments 1 to 8, wherein the polynucleotide element of the genome-editing system is introduced into callus comprising the plant cell that expresses the polypeptide element(s).
10. The method of any one of embodiments 1 to 8, wherein the polynucleotide element of the genome-editing system is introduced into an explant comprising the plant cell that expresses the polypeptide element(s).
11. The method of any one of embodiments 1 to 10, wherein the time between the introducing of step (a) and the introducing of step (c) is: (i) at least about 72 hours; or (ii) about 72, 84, or 96 hours to about 108, 120, or 144 hours.
12. The method of any one of embodiments 1 to 10, wherein the time between the introducing of step (a) and the introducing of step (c) is about 4 weeks to about 5 weeks.
13. The method of any one of embodiments 1 to 12, wherein the polynucleotide encoding the one or more polypeptide elements of the genome-editing system comprises a first selectable marker.
14. The method of embodiment 13, wherein the first selectable marker comprises a phosphinothricin acetyltransferase marker or an imazapyr tolerance marker.
15. The method of any one of embodiments 1 to 14, wherein the one or more polynucleotide elements of a genome-editing system comprise a second selectable marker.
16. The method of embodiment 15, wherein the second selectable marker comprises an imazapyr tolerance marker, a phosphinothricin acetyltransferase marker, or a fluorescent marker.
17. The method of any one of embodiments 1 to 16, wherein the polypeptide elements of the genome-editing system further comprise one or more homology dependent repair (HDR)-enhancing polypeptides, optionally wherein the HDR-enhancing polypeptides comprise a bacteriophage lambda exonuclease, a bacteriophage lambda beta single-stranded annealing protein (SSAP), and/or an E. coli single stranded binding protein (SSB).
18. The method of any one of embodiments 1 to 17, wherein the plant cell is a maize plant cell or a soybean plant cell.
19. A method of producing a genome edited plant callus comprising: (a) introducing into a plant cell a polynucleotide encoding one or more polypeptide element(s) of a genome-editing system; (b) selecting callus that expresses the polypeptide element(s), wherein the callus is obtained from the plant cell of step (a); (c) introducing into cells of the callus that expresses the polypeptide element(s) one or more polynucleotide elements of the genome-editing system; and (d) identifying a genome edited plant callus comprising a genome edited plant cell from the plant cell obtained in step (c).
20. The method of embodiment 19, further comprising regenerating a genome edited plant from the genome edited callus of step (d).
21. The method of embodiment 19 or embodiment 20, wherein the callus that expresses the polypeptide element(s) in step (b) has not been obtained from a regenerated plant that expresses the polypeptide element(s).
22. The method of any one of embodiments 19 to 21, wherein the polypeptide element of the genome-editing system comprises an RNA-guided nuclease.
23. The method of any one of embodiments 19 to 22, wherein the polynucleotide element of the genome-editing system comprises a donor template polynucleotide.
24. The method of any one of embodiments 19 to 23, wherein the polynucleotide element of the genome-editing system comprises a guide RNA (gRNA) for use with the RNA-guided nuclease, or a DNA encoding a gRNA for use with the RNA-guided nuclease.
25. The method of any one of embodiments 19 to 24, wherein the introducing of step (a) is by Agrobacterium-mediated transformation and optionally wherein the introducing of step (c) is by biolistics.
26. The method of any one of embodiments 19 to 25, wherein the introducing of step (c) is by biolistics.
27. The method of any one of embodiments 19 to 26, wherein the time between the introducing of step (a) and the introducing of step (c) is: (i) at least about 72 hours; or (ii) about 72, 84, or 96 hours to about 108, 120, or 144 hours.
28. The method of any one of embodiments 19 to 26, wherein the time between the introducing of step (a) and the introducing of step (c) is about 4 weeks to about 5 weeks.
29. The method of any one of embodiments 19 to 26, wherein the polynucleotide encoding the one or more polypeptide elements of the genome-editing system comprises a first selectable marker.
30. The method of embodiment 29, wherein the first selectable marker comprises a phosphinothricin acetyltransferase marker or an imazapyr tolerance marker.
31. The method of any one of embodiments 19 to 30, wherein the one or more polynucleotide elements of a genome-editing system comprise a second selectable marker.
32. The method of embodiment 31, wherein the second selectable marker comprises an imazapyr tolerance marker, a phosphinothricin acetyltransferase marker, or a fluorescent marker.
33. The method of any one of embodiments 19 to 32, wherein the polypeptide elements of the genome-editing system further comprise one or more homology dependent repair (HDR)-enhancing polypeptides, optionally wherein the HDR-enhancing polypeptides comprise a bacteriophage lambda exonuclease, a bacteriophage lambda beta single-stranded annealing protein (SSAP), and/or an E. coli single stranded binding protein (SSB).
34. The method of any one of embodiments 19 to 33, wherein the plant cell is a maize plant cell or a soybean plant cell.

### EXAMPLES

### Example 1

The approach relies on performing an Agrobacterium mediated transformation using a Cas containing cassette and selection marker as vector. The first step involves a standard Agrobacterium transformation protocol and consists of obtaining selected callus that expresses the Cas enzyme in most cells. The next step uses a biolistics transformation protocol with guide(s), oligos, and a different selection marker on the callus obtained in the first step, the cells of which are all already stably expressing the Cas enzyme.

Two versions of the method were tested: A fast method (embryo bombardment) that included Agrobacterium transformation delivery of Cas followed by biolistics on transient Cas-expressing embryos to deliver gRNA, oligos, and plasmid for selection; and a slow method (callus bombardment) that included Agrobacterium transformation delivery of Cas followed by biolistics on Cas-expressing callus to deliver gRNA, oligos, and plasmid for selection.

Immature B104 embryos were transformed by Agrobacterium (LBA4404) mediated transformation. The T-DNAs had either a blunt-end cutting Cas nuclease-encoding sequence or a staggered-end cutting Cas nuclease-encoding sequence downstream of a ZmUbi1 promoter between the left and right borders, as well as the PAT selection marker.

After co-incubation with Agrobacterium, callus formation was promoted by after 3 days. The embryos were placed on callus initiation medium (BOM1; Table 1) for 3 days and then the embryos were transferred to osmosis medium (BOM2; Table 2) for 4 hours and 15 minutes before bombardment.

Callus was then transformed by biolistics. The bombarded nucleic acids included an expression plasmid having expression cassettes for an mScarlett fluorescent marker and a ZmAHAS imazapyr selection marker, as well as a nuclease guide and double stranded oligonucleotides for NHEJ insertion at the cut sites.

For the blunt-end cutting Cas tests, a single guide targeted TGGCTTTTGCTGAGGGGATA (SEQ ID NO: 1) of the corn genome (ZmGS2). For the staggered-end cutting Cas tests the guide also targeted ZmGS2. The 2PS ds oligos were G*T*AAGCGCTTACGTAAGCGCTTACGTAAGCGCTT*A*C (SEQ ID NO: 2; the asterisks indicate phosphorothioate bonds modifications). Genotyping of the treated callus was performed by DNA extraction from callus and ampseq for the target locus.

TABLE 1
BOM1 media (isolation and callus initiation).

| Component | Manufacturer-product | | Units/L |
|---|---|---|---|
| MS medium | Duchefa-M0221 | 4.3 | g |
| myo inositol | Duchefa-I0609 | 0.1 | g |
| L-Proline | Duchefa-P0717 | 2.76 | g |
| Sucrose | Duchefa-S0809 | 30 | g |
| Casein hydrolysate | Duchefa-C1301 | 0.1 | g |
| pH | | 5.8 | |
| Gelrite | Duchefa-G1101 | 3 | g |
| | | Autoclave | |
| Dicamba (6.63 g/L) | Duchefa-D0920 | 0.5 | mL |
| Silver nitrate (100 mM) | Sigma-S7276 | 0.9 | ml |
| MS vitamins (1000x stock) | Duchefa-M0409 | 1 | ml |

TABLE 2
BOM2 media (osmosis).

| Component | Manufacturer-product | | Units/L |
|---|---|---|---|
| MS medium | Duchefa-M0221 | 4.3 | g |
| myo inositol | Duchefa-I0609 | 0.1 | g |
| L-Proline | Duchefa-P0717 | 0.69 | g |
| Sucrose | Duchefa-S0809 | 30 | g |
| Casein hydrolysate | Duchefa-C1301 | 0.1 | g |
| D-Sorbitol | Duchefa-S0807 | 36.4 | g |
| D-Mannitol | Duchefa-M0803 | 36.4 | g |
| pH | | 5.8 | |
| Gelrite | Duchefa-G1101 | 3 | g |
| | | Autoclave | |
| Dicamba (6.63 g/L) | Duchefa-D0920 | 0.5 | mL |
| Silver nitrate (100 mM) | Sigma-S7276 | 0.9 | mL |
| MS vitamins (1000x stock) | Duchefa-M0409 | 1 | mL |

### Blunt-End Cutting Results

Embryo bombardment: across 4_experiments, there were reads with insertions in about 13% of calli, but <1% of total reads.

Callus bombardment: across 3 experiments, there were reads with insertions in about 5% of calli, at up to 1.1% of total reads; total editing up to 8%.

### Staggered-End Cutting Results

Embryo bombardment: across 2 experiments, 9% of calli with reads with insertions, up to 2% of total reads.

Callus bombardment: across 3 experiments, 10-20% of calli had efficient editing at T-DNA encoded gRNA; 2% of calli with reads with insertions, up to 1.8% of total reads; total editing up to 7.3%.

The breadth and scope of the present disclosure should not be limited by any of the above-described examples.

What is claimed is:

1. A method of producing a genome edited plant cell comprising:
(a) introducing into a plant cell a polynucleotide encoding one or more polypeptide element(s) of a genome-editing system;
(b) selecting for a plant cell that expresses the polypeptide element(s);
(c) introducing one or more polynucleotide element(s) of the genome-editing system into the plant cell that expresses the polypeptide element(s); and
(d) identifying a genome edited plant cell from the plant cell obtained in step (c).

2. The method of claim 1, further comprising the step of regenerating a genome edited plant from the genome edited plant cell of step (d).

3. The method of claim 1, wherein the plant cell that expresses the polypeptide element(s) in step (c) has not been obtained from a regenerated plant that expresses the polypeptide element(s).

4. The method of claim 1, wherein the polypeptide element of the genome-editing system comprises an RNA-guided nuclease.

5. The method of claim 1, wherein the polynucleotide element of the genome-editing system comprises a donor template polynucleotide and/or wherein the polynucleotide element of the genome-editing system comprises a guide RNA (gRNA) for use with the RNA-guided nuclease, or a DNA encoding a gRNA for use with the RNA-guided nuclease.

6. (canceled)

7. The method of claim 1, wherein the introducing of step (a) is by Agrobacterium-mediated transformation and optionally wherein the introducing of step (c) is by biolistics.

8. (canceled)

9. The method of claim 1, wherein the polynucleotide element of the genome-editing system is introduced into callus comprising the plant cell that expresses the polypeptide element(s).

10. The method of claim 1, wherein the polynucleotide element of the genome-editing system is introduced into an explant comprising the plant cell that expresses the polypeptide element(s).

11. The method of claim 1, wherein the time between the introducing of step (a) and the introducing of step (c) is: (i) at least about 72 hours; (ii) about 72, 84, or 96 hours to about 108, 120, or 144 hours; or (iii) about 4 weeks to about 5 weeks.

12. (canceled)

13. The method of claim 1, wherein the polynucleotide encoding the one or more polypeptide elements of the genome-editing system comprises a first selectable marker

14. (canceled)

15. The method of claim 1, wherein the one or more polynucleotide elements of a genome-editing system comprise a second selectable marker.

16. (canceled)

17. The method of claim 1, wherein the polypeptide elements of the genome-editing system further comprise one or more homology dependent repair (HDR)-enhancing polypeptides, optionally wherein the HDR-enhancing polypeptides comprise a bacteriophage lambda exonuclease, a bacteriophage lambda beta single-stranded annealing protein (SSAP), and/or an E. coli single stranded binding protein (SSB).

18. The method of claim 1, wherein the plant cell is a maize plant cell or a soybean plant cell.

19. A method of producing a genome edited plant callus comprising:
(a) introducing into a plant cell a polynucleotide encoding one or more polypeptide element(s) of a genome-editing system;
(b) selecting callus that expresses the polypeptide element(s), wherein the callus is obtained from the plant cell of step (a);
(c) introducing into cells of the callus that expresses the polypeptide element(s) one or more polynucleotide elements of the genome-editing system; and
(d) identifying a genome edited plant callus comprising a genome edited plant cell from the plant cell obtained in step (c).

20. The method of claim 19, further comprising regenerating a genome edited plant from the genome edited callus of step (d).

21. The method of claim 19, wherein the callus that expresses the polypeptide element(s) in step (b) has not been obtained from a regenerated plant that expresses the polypeptide element(s).

22. The method of claim 19, wherein the polypeptide element of the genome-editing system comprises an RNA-guided nuclease.

23. The method of claim 19, wherein the polynucleotide element of the genome-editing system comprises a donor template polynucleotide and/or the polynucleotide element of the genome-editing system comprises a guide RNA (gRNA) for use with the RNA-guided nuclease, or a DNA encoding a gRNA for use with the RNA-guided nuclease.

24. (canceled)

25. The method of claim 19, wherein the introducing of step (a) is by Agrobacterium-mediated transformation and optionally wherein the introducing of step (c) is by biolistics.

26. (canceled)

27. The method of claim 19, wherein the time between the introducing of step (a) and the introducing of step (c) is: (i) at least about 72 hours; (ii) about 72, 84, or 96 hours to about 108, 120, or 144 hours; or (iii) about 4 weeks to about 5 weeks.

28-32. (canceled)

33. The method of claim 19, wherein the polypeptide elements of the genome-editing system further comprise one or more homology dependent repair (HDR)-enhancing polypeptides, optionally wherein the HDR-enhancing polypeptides comprise a bacteriophage lambda exonuclease, a bacteriophage lambda beta single-stranded annealing protein (SSAP), and/or an E. coli single stranded binding protein (SSB).

34. The method of claim 19, wherein the plant cell is a maize plant cell or a soybean plant cell.
